(19) United States
(12) Patent Application Publication
(10) Pub. No.: US 2017/0001025 A1
(43) Pub. Date: Jan. 5, 2017

(54) AESTHETIC METHOD OF BIOLOGICAL STRUCTURE STIMULATION BY MAGNETIC FIELD

(71) Applicant: BTL HOLDINGS LIMITED, Limassol (CY)

(72) Inventors: Tomás Schwarz, Prague (CZ); Ondra Prouza, Ricany u Prahy (CZ)

(21) Appl. No.: 14/951,093

(22) Filed: Nov. 24, 2015

Related U.S. Application Data
(63) Continuation-in-part of application No. 14/926,365, filed on Oct. 29, 2015.
Application No. 14/926,365 is a continuation-in-part of application No. 14/789,156, filed on Jul. 1, 2015, now abandoned.
Application No. 14/789,156 is a continuation-in-part of application No. 14/789,658, filed on Jul. 1, 2015.

(51) Int. Cl. A61N 2/00

(52) U.S. Cl. CPC A61N 2/004

(57) ABSTRACT

In a method for stimulation and treatment of a biological structure the biological structure is stimulated by high power time-varying magnetic field. The stimulation is followed by at least a partial muscle contraction. The method may be used e.g. in applications for non-invasive aesthetic medicine.

### PRIORITY CLAIM

This application is a continuation-in-part of U.S. patent application Ser. No. 14/926,365 filed Oct. 30, 2015 and now pending, which is a continuation-in-part of U.S. patent application Ser. No. 14/789,156 filed Jul. 1, 2015, and now pending. This Application is also a Continuation-in-Part of U.S. patent application Ser. No. 14/789,658 filed Jul. 1, 2015, and now pending. application Ser. Nos. 14/926,365; 14/789,156 and 14/789,658 are incorporated herein by reference.

### FIELD OF THE INVENTION

The present invention generally relates to methods using the influence of magnetic and induced electric field on biological structure. The magnetic field is time-varying and high powered therefore the method is based on a value of magnetic flux density sufficient to induce at least partial muscle contraction.

### BACKGROUND OF THE INVENTION

Aesthetic medicine comprises all medical procedures that are aimed at improving the visual appearance and satisfaction of the patient. Patients not only want to be in good health, they also want to minimize all imperfections including body shape and effects of natural aging. Indeed, patients request quick, non-invasive procedures providing satisfactory results with minimal risks.

The most common methods used for non-invasive aesthetic medicine applications are based on application of mechanical waves, e.g. ultrasound or shock wave therapy; or electromagnetic waves, e.g. radiofrequency treatment or light treatment, such as intense pulsed light or laser treatment. The effect of mechanical waves on tissue is based especially on cavitation, vibration and/or heat inducing effects. The effect of applications using electromagnetic waves is based especially on heat production in the biological structure.

Skin tissue is composed of three basic elements: epidermis, dermis and hypodermis or so called subcutis. The outer and also the thinnest layer of skin is the epidermis. The dermis consists of collagen, elastic tissue and reticular fibres.

The hypodermis is the lowest layer of the skin and contains hair follicle roots, lymphatic vessels, collagen tissue, nerves and also fat forming a subcutaneous white adipose tissue (SWAT). The fat cells create lobules which are bounded by connective tissue, fibrous septa (retinaculum cutis).

Another part of adipose tissue, so called visceral fat, is located in the peritoneal cavity and forms visceral white adipose tissue (VWAT) located between parietal peritoneum and visceral peritoneum, closely below muscle fibres adjoining the hypodermis layer.

Water and biological molecules are diamagnetic substances. The magnetic field is not affected by diamagnetic substances. Therefore no loss of intensity or magnetic flux density occurs when passing through the biological structure or tissue. Therefore the deep biological structures may be stimulated by the time-varying magnetic field as well. One of the time-varying magnetic fields is the so called pulsed electromagnetic field (PEMF). PEMF is limited by the repetition rate and even by the magnetic flux density. PEMF repetition rates are in range of 5 to 100 Hz and magnetic flux density is up to 600 Gauss (equivalent to 60 mT) based on Physikalische Medizin. [HEISEL, Jürgen. Physikalische Medizin. Stuttgart: Georg Thieme Verlag KG, 2005. ISBN 3-13-139881-7. p. 159]. Another source recites the highest magnetic flux density of 6 mT. [BRONZINO, Joseph, D. The Biomedical Engineering Handbook, Volume I. United States of America: CRC Press LLC, 2000. Second edition. ISBN 0-8493-0461-X. p. 91-1-91-8].

PEMF may be used to improve the conventional non-invasive aesthetic medicine applications, e.g. radiofrequency treatment, diathermy treatment, ultrasound treatment, etc. The enhanced visual results are achieved in combination with these methods as described in patent application US2015025299. The magnetic flux density of the recited magnetic field reaches 20 Gauss and the repetition rate varies from 16 to 25 Hz.

US2015157873 recites an applicator including stimulation coil for emitting magnetic field to the patient to induce muscle contraction for the purpose of fatty tissue breakdown. The magnetic flux density is in range of 0.01 to 0.1 T at distance of 5 cm from the coil surface, the recited repetition rate varies from 10 to 30 Hz and impulse duration is in range of 100 to 300 μs. However, the proposed stimulation by magnetic flux density 0.1 T is limited to stimulate only superficial muscle. Additionally, this stimulation may be time consuming and provide limited results. Magnetic flux density of 0.1 T is insufficient to intensively stimulate superficial muscle or deep biological structures since the magnetic flux density is low. The magnetic flux density is able to stimulate limited number of muscle fibres. Therefore the muscle contraction is weak and the method is insufficient to be applied for satisfactory enhancement of visual appearance. In order to achieve any results the treatment duration lasts long time period. Accordingly, patients with a high BMI cannot be well treated using existing methods.

Present aesthetic approaches do not propose any method sufficient for visual enhancement by contact and/or contactless use. The above-described methods are limited in key parameters which are repetition rate and/or magnetic flux density. All known methods use low values of magnetic flux density and/or low repetition rates which does not allow satisfactory enhancement of visual appearance. As a result, new methods are needed to enhance the visual appearance of the patient.

### SUMMARY OF THE INVENTION

In a first aspect, a method for stimulation of biological structure uses a time-varying magnetic field at a magnetic flux density of at least 0.15 T and/or repetition rate 30 Hz for the purpose of at least a partial muscle contraction and enhancing the visual appearance of the treated body area.

In another aspect, the stimulation utilizes non-invasive and/or contactless transfer of the stimulation signal from an applicator to biological structure to evoke the action potential of the biological structure to induce at least partial muscle contraction. The applicator may include a source of magnetic field e.g. a coil.

The present methods enable new treatments defined by a peak to peak magnetic flux density on the coil surface at least 0.15, 0.2, 0.8, 1.5, 2, 2.4 or up to 7 Tesla at repetition rate at least 1, 10, 30, 50, 55, 60 or up to 700 Hertz with treatment/successive treatments lasting several seconds or longer, for example, for at least 5, 10, 30, 60, 120 or 240 seconds, or longer. The pulse width is in the range of tens to hundreds of μs.

In further aspect, a neuromuscular plate and/or the nerve innervating the neuromuscular plate is stimulated and at least partial muscle contraction is provided. The muscle may be contracted at higher repetition rates and the contraction is stronger. Therefore the stimulation is more efficient for reducing the number and/or volume of adipocytes and enhancing the visual appearance of the treated body area via targeted muscle contraction. Additionally, strong muscle contractions at higher repetition rates cause mechanical movement of all the layers in proximity of the contracted muscle. This method therefore causes remodeling and/or neogenesis of the collagen and elastin fibres.

According to another embodiment the method may be applied for enhancing the visual appearance of body area including or proximate to major muscle structures. Further the method for enhancing the visual appearance is convenient for treatment of patients with high BMI.

The muscle tissue is selectively stimulated and the magnetic flux density of the stimulation may be adjusted based on the patient's feeling and/or needs. Treatment time is shortened due to selective stimulation of muscles. Additionally, the treatment may be non-invasive or even contactless due to the high value of magnetic flux density. Invasive methods may be used as well. The patient may be treated without the necessity of taking clothes off. The method reduces patient discomfort.

The present methods may be used for enhancing visual appearance of body areas including adipose tissue reduction, muscle toning, muscle shaping, body contouring, body shaping, skin tightening, cellulite treatment, circumferential reduction, breast enhancement and/or lip enhancement.

GLOSSARY

Conventional non-invasive and/or invasive aesthetic medicine applications refer to aesthetic medicine applications based on application of mechanical waves, e.g. ultrasound or shock wave therapy; or electromagnetic waves, e.g. radiofrequency or diathermy treatment or light treatment, such as intense pulsed light or laser treatment; or mechanical stimulation, e.g. positive or negative pressure, rollerball, massage etc.; or thermal treatment, e.g. cryotherapy; or electrotherapy method; or mesotherapy method and or any combination thereof.

Biological structure/target biological structure includes cells, neurons, nerves, muscle fibres and/or muscle tissue.

Stimulation signal refers to a magnetic flux density inducing an electric current in the biological structure.

Body area includes skin, muscle fibre, muscle or muscle group, collagen, elastin, adipose cell or tissue, limb and/or any other tissue.

Muscle includes at least one of muscle fibre, muscle tissue or group, neuromuscular plate or nerve innervating the at least one muscle fibre.

Deep muscle refers to a muscle that is at least partly below superficial muscles and/or to the muscle that is covered by the thick layer of other tissue, e.g. mostly adipose tissue and/or the skin, with thickness 0.5, 1, 2, 3, 4, 5 or more centimetres.

Impulse refers to the only one biphasic magnetic stimulus.

Pulse refers to a period of stimulation signal of at least one biphasic magnetic stimulus and time duration of no stimulation, i.e. time duration between two impulses from rise edge to next rise edge.

Repetition rate refers to frequency of firing the pulses; it is derived from the time duration of a pulse.

### DETAILED DESCRIPTION

Electric current is induced in the stimulated biological structure during pulsed magnet treatment. Due to the high value of magnetic flux density the biological structure may be targeted and stimulated more specifically. A distribution of magnetic field is uniform in the biological structure. Particles (e.g. atoms, ions, molecules etc.) in the biological structures are influenced by the magnetic field and permeability of a cell membrane also increases.

Due to increased permeability of the cell membrane, the pulsed magnet treatment effects especially these processes and can induce following effects: at least partial muscle contraction; reduction of adipose tissue—volume and/or number of the adipose cells; neogenesis and/or remodeling of collagen and/or elastin fibres; improving circulation of blood and/or lymph and improves local and/or adipose tissue metabolism.

With the present methods, factors for enhancing visual appearance of the body are: stimulation of major muscle, e.g. gluteus maximus; stimulation of deep muscle which is enabled by high value of magnetic flux density; non-contact application of magnetic flux density, it may be applied even through clothing; stronger muscle contraction due to higher value of magnetic flux density; higher-quality of muscle targeting; treatment is not influenced by small movements during treatment; treatment time duration is shortened due to high value of magnetic flux density and/or higher repetition rate; no delays occur.

It is to be understood that the method is not limited to the particular applications and that the method may be practiced or carried out in various ways.

Present method is especially applied for enhancing the visual appearance of body parts including or proximate to major muscle structures. Further the method is applicable for enhancing the visual appearance of patients with high value of BMI. The present method is not limited to the application of the stimulation signal to major muscle. Muscles other than major muscles may be stimulated as well.

The applicator of magnetic stimulation signal is placed proximate to the patient's body. As used here, proximate to includes both contactless and in actual contact with the skin of the patient. The muscles are selectively stimulated by the stimulation signal and the magnetic flux density of the stimulation may be adjusted following the patient's feeling or needs. The treatment time is shortened due to selective stimulation of the correct muscles. Additionally, due to the high value of magnetic flux density, the muscle is stimulated more effectively. Further, the treatment may be non-invasive or even preferably contactless due to the high value of magnetic flux density. The patient may be treated without removing clothing, reducing patient discomfort. Additionally, following the high efficiency of the muscle contraction the collagen and/or elastin fibres above the muscle structure are remodeled, hence the visual appearance is enhanced.

The magnetic stimulation of the biological structure has various applications for enhancing visual appearance of the contour body area. High density magnetic field reaches such values which may be used for: adipose tissue reduction, wherein the adipose tissue reduction is achieved by reduction of number and/or volume of adipose cells; muscle toning, wherein the muscle appearance enhancement is achieved by adipose tissue reduction with no muscle bulking; muscle shaping, wherein the muscle appearance enhancement is achieved by adipose tissue reduction and/or muscle bulking; body contouring, wherein the silhouette appearance enhancement is achieved by adipose tissue reduction with no muscle bulking; body shaping, wherein the silhouette appearance enhancement is achieved by adipose tissue reduction and/or muscle bulking; skin tightening, wherein the skin appearance enhancement is achieved by obtaining smoother and younger appearance, including wrinkles reduction; cellulite treatment, wherein the appearance enhancement is achieved by adipose tissue reduction, muscle contraction and/or elastic fibres neogenesis; circumferential reduction, wherein the reduction is achieved by adipose tissue reduction and/or the muscle bulking; breast enhancement, wherein the appearance enhancement effect is achieved by elevation or shape modification; lip enhancement, wherein the lip appearance enhancement is achieved by obtaining fuller and firmer appearance.

In the methods described, the magnetic stimulation device may or may not include a magnetic core. The magnetic stimulation device may be cooled by fluid, e.g. by air. Total power consumption of the magnetic stimulation device may be below 1.3 kW. A magnetic stimulation device as described in the U.S. patent application Ser. No. 14,789,156 or U.S. patent application Ser. No. 14,789,658, incorporated herein by reference, may be used.

The applicator for magnet treatment is placed proximate to the patient's body. The magnetic flux density is applied into the target biological structure. Electric current is induced and stimulates the neuromuscular plate and/or the nerve innervating the at least one muscle fibre. The stimulation causes at least a partial muscle contraction.

The present method stimulates the biological structure by pulsed magnetic field. The peak to peak magnetic flux density on the coil surface is at least 0.15, 0.2, 0.8, 1.5, 2, 2.4 or up to 7 Tesla at repetition rate at least 1, 10, 30, 50, 55, 60, or up to 700 Hertz with treatment/successive treatments lasting several seconds or longer, for example, for at least 5, 10, 30, 60, 120 or 240 seconds, or longer. The pulse width is in the range of tens to hundreds of μs.

Cellulite is an effect of skin change resulting in orange peel appearance. The cause of the cellulite is orientation of collagen fibres in so called “fibrous” septae. The fibrous septae contract and harden over time creating a dimple effect. Additionally, blood and lymphatic vessels lack circulation due to the contraction and hardening of the septae. The lymph flow may be blocked resulting in swelling. Another cause of cellulite is adipose cells protruding to dermis.

One application of time-varying magnetic field for enhancing the visual appearance of body area may be stimulation of a muscle by magnetic flux density for reducing the cellulite. The magnetic flux density is delivered through the skin to the neuromuscular plate and/or nerve innervating at least one muscle fibre. The electric current is induced in the target biological structure causing at least partial muscle contraction. The at least partial muscle contraction causes the movement of the skin and all the biological structures subtending epidermis. Additionally, the at least partial muscle contraction improves blood circulation by itself, or via the movement of the muscle in the vicinity including fibrous septae. Additionally, blood and/or lymph circulation is improved in the layers subtending epidermis since the muscle contraction moves the fibrous septae. Also local and/or adipose tissue metabolism is improved. The at least partial muscle contraction is more effective for adipose tissue metabolism as the value of magnetic flux density increases since the muscle contraction is stronger. The higher magnetic flux density effects the higher number of muscle fibres contraction and the more adipose tissue is reduced. Therefore the visual appearance of regions prone to cellulite is enhanced.

The method causes the circumferential reduction i.e. a reduction of the size of the treated body area. The method is mostly indicated for the regions with cellulite, especially for buttocks, abdomen, hips, thighs or arms. However, the indication is not limited to the mentioned regions and the method may be used for stimulation of any other body area.

The present method may provide a massage effect via the stimulation which is caused by the at least partial muscle contraction. Therefore the massage effect may be achieved by contactless methods instead of manual massage techniques or soft tissue techniques. The massage effect improves lymph circulation.

In another aspect, improvement of functionality and/or the appearance of the muscle is achieved with results similar to body exercise. The results are achieved by application of high magnetic flux density to the body area and inducing at least partial muscle contraction. Higher values of magnetic flux density applied result in a stronger muscle contraction. The patient feels firmer and tighter.

With the present method muscle contractions induced by the applied magnetic flux density help to tone the muscle providing a more attractive appearance. As the muscle structure is stimulated by time-varying magnetic field the entire limb may be moved due to the high power of the magnetic stimulation signal. Nevertheless, the method is not limited to the applications to the limbs and the method is able to be applied to stimulation of any muscle, e.g. gluteus maximus or any muscle/deep muscle to induce body contouring and/or body shaping effect and fat burn. Additionally, shortened and/or flabby muscles are stretched. The physical fitness of the patient is improved as well.

The present methods may also induce muscle contraction to reduce effect of skin laxity. Skin laxity may be caused by e.g. the aging process or increasing number and/or volume of adipose cells which pulls down the skin by gravity, rapid weight loss or skin stretching during the pregnancy. The muscles are stimulated by the induced electric current to contract. Repetitive contractions cause the muscles to obtain the tonus and flexibility. Therefore the skin appearance is enhanced by stimulating the flabby muscles. The effect of skin tightening is achieved. The method also stimulates the creation of the collagen and elastin fibres in the layers subtending the epidermis hence the skin obtains enhanced visual appearance. The method may be widely applied but not limited to application to the regions of neck, breasts, arms or abdomen. The method provides the smoother and younger appearance of the skin to the patient.

Similar methods of stimulation the muscle structure by time-varying magnetic field for inducing the at least partial muscle contraction may be used for treatment of wrinkles as well. Wrinkles are results of extrinsic and intrinsic factors. Nowadays, wrinkles are considered to be negative effect of natural aging process which decreases the production of collagen and elastin fibres and weakens the skin which becomes thinner. As the muscle stimulation by the magnetic flux density induces at least partial muscle contraction, the stimulation of collagen and elastin fibres neogenesis is improved. Additionally, the muscles subtending the stimulated region are toned and the skin gets a younger and enhanced visual appearance. Therefore, the effect of skin tightening is achieved.

Wrinkles may be prevented or reduced by practising facial exercises which causes a massage effect to the facial tissues, improving blood and lymph circulation. Additionally, the facial muscles are relaxed and toned after the exercise. A similar effect as facial exercise may be achieved by non-invasive and/or contactless method of stimulating the facial muscles by magnetic flux density. Further additional advantage of the present method is the improvement of restoration of the collagen and elastin fibres, more effective toning and strengthening of the facial muscles.

The present methods may improve the neogenesis and remodeling of collagen fibres in the lips to reach a full, plump and firmer appearance. The magnetic flux density is applied to the lips by an applicator. Therefore the lips become fuller and firmer without any need of invasive method such as injection of the synthetic fillers, permanent makeup or the facial implants. The present method stimulates the remodeling and/or neogenesis of collagen fibres in a natural way. Additionally, the collagen is natural substance of the human body which provides the elasticity to the structure.

The present methods may be used for enhancing the visual appearance of breasts. Cooper's ligament may be stimulated, improved and/or firmed by the at least partial muscle contraction. The muscle stimulation induces the elevation of the breast tissue. Additionally, the breast tissue is stimulated to be modified in a shape, wherein the shape includes the size and/or the contour of the breast tissue. Therefore the visual appearance is enhanced and breasts are more attractive for the patient. The present method is a non-invasive alternative for current aesthetic surgery method for the treatment of sagging breast tissue. The present method provides a patient a method of breast visual appearance enhancement without surgery. Therefore the method lacks post-surgery complications such as scars, postoperative pain or long recovery period. Various treatment protocols may be used.

Following the recited methods the stimulation signal may be but is not limited to continuous, pulsed, randomized or burst. The impulse may be but not limited to monophasic, polyphasic and/or biphasic.

In the preferred application of the present method the trains of pulses, called bursts are used.

Repetition rate and/or magnetic flux density may vary during the treatment protocol. Further the magnetic stimulation signal may include several periods of stimulation signal of different repetition rates, therefore the modulation of the signal is in repetition rate domain. The stimulation signal may include several periods of stimulation signal of different magnetic flux densities, therefore the modulation of the signal is in magnetic flux density domain. In yet another approach the envelope of the stimulation signal may be modulated by combinations of repetition rate domain and magnetic flux density domain.

Various envelopes of the stimulation signal and waveform, e.g. pulse, sinusoidal, rectangular, square, triangular, saw-tooth, trapezoidal, exponential etc. for the purpose of muscle stimulation may also be used, and is not limited to recited shapes of stimulation signals.

The values of magnetic flux density and repetition rate are cited in several preferred applications since the perception of the stimulation is subjective. Nevertheless, the magnetic flux density and repetition rates are not limited by the recited values. A person skilled in the physical therapy is able to repeat and apply the treatment methods adjusting the magnetic flux density and/or repetition rate following the patient's sensitivity or needs.

The present method is not limited to be used independently. For enhancing the result the method may be used in combination with other conventional non-invasive and/or invasive aesthetic medicine method.

All the recited methods may be applied to a patient in a non-invasive and/or contactless way. Therefore the present methods provide an effective alternative approach of enhancing the visual appearance with no need of invasive treatment or surgery. Further, the visual results are appreciable after several treatments. Additionally, the results include not only the visual appearance enhancement but even the improvement of the muscle structures, hence the patient feels firmer and tighter. The muscle structures become toned with no need of any diet or spending time by exercising in fitness.

Thus, novel systems and methods have been described. Various changes and substitutions may of course be made without departing from the spirit and scope of the invention. The invention, therefore, should not be limited, except by the following claims and their equivalents.

1. A method for enhancing an appearance of a patient's body, comprising:
placing a magnetic applicator proximate to at least a part of the patient's body;
applying a time-varying magnetic stimulation signal to a target biological structure to induce electric current in the target biological structure wherein the target biological structure is a muscle and wherein the induced electric current causes an at least partial contraction of the muscle, providing an enhanced appearance of the patient's body.

2. The method of claim 1, wherein the time-varying magnetic stimulation signal provides a magnetic flux density of at least 0.15 T.

3. The method of claim 1, wherein the time-varying magnetic stimulation signal provides a magnetic flux density of at least 0.2 T.

4. The method of claim 1, wherein the time-varying magnetic stimulation signal provides a magnetic flux density of at least 0.8 T.

5. The method of claim 1, wherein the time-varying magnetic stimulation signal provides a magnetic flux density of at least 2 T.

6. The method of claim 1, wherein the time-varying magnetic stimulation signal provides a magnetic flux density of at least 2.4 T.

7. The method of claim 1, wherein the time-varying magnetic stimulation signal provides a magnetic flux density of at least 7 T.

8. The method of claim 2, wherein the time-varying magnetic stimulation signal is applied with a repetition rate of at least 1 Hz.

9. The method of claim 2, wherein the time-varying magnetic stimulation signal is applied with a repetition rate of at least 10 Hz.

10. The method of claim 2, wherein the time-varying magnetic stimulation signal is applied with a repetition rate of at least 30 Hz.

11. The method of claim 2, wherein the time-varying magnetic stimulation signal is applied with a repetition rate of at least 50 Hz.

12. The method of claim 2, wherein the time-varying magnetic stimulation signal is applied with a repetition rate of at least 55 Hz.

13. The method of claim 1, wherein the time-varying magnetic stimulation signal is applied with a repetition rate of at least 30 Hz.

14. The method of claim 1, wherein the time-varying magnetic stimulation signal is applied with a repetition rate of at least 50 Hz.

15. The method of claim 1, wherein the time-varying magnetic stimulation signal is applied with a repetition rate of at least 55 Hz.

16. The method of claim 1, wherein the time-varying magnetic stimulation signal is applied with a repetition rate of at least 700 Hz.

17. The method of claim 1, wherein the stimulation lasts at least 5 seconds.

18. The method of claim 1, wherein the stimulation signal is modulated in a repetition rate domain and/or a magnetic flux density domain.

19. The method of claim 1, the at least partial contraction of the muscle leads to at least one of adipose tissue reduction, muscle toning, muscle shaping, body contouring, body shaping, skin tightening, cellulite treatment, circumferential reduction, breast enhancement and/or lip enhancement.

20. A method for enhancing an appearance of a patient's body, comprising:
placing a magnetic applicator proximate to at least a part of the patient's body;
applying a time-varying magnetic stimulation signal to a target biological structure to induce electric current in the target biological structure wherein the target biological structure is a muscle and wherein the induced electric current causes an at least partial deep contraction occurs contraction of a deep muscle, providing an enhanced appearance of the patient's body.

21. The method of claim 20, wherein the time-varying magnetic stimulation signal provides a magnetic flux density of at least 0.15 T.

22. The method of claim 20, wherein the time-varying magnetic stimulation signal provides a magnetic flux density of at least 0.2 T.

23. The method of claim 20, wherein the time-varying magnetic stimulation signal provides a magnetic flux density of at least 0.8 T.

24. The method of claim 20, wherein the time-varying magnetic stimulation signal provides a magnetic flux density of at least 2 T.

25. The method of claim 20, wherein the time-varying magnetic stimulation signal provides a magnetic flux density of at least 2.4 T.

26. The method of claim 20, wherein the time-varying magnetic stimulation signal is applied with a repetition rate of at least 1 Hz.

27. The method of claim 20, wherein the time-varying magnetic stimulation signal is applied with a repetition rate of at least 10 Hz.

28. The method of claim 20, wherein the time-varying magnetic stimulation signal is applied with a repetition rate of at least 30 Hz.

29. The method of claim 20, wherein the time-varying magnetic stimulation signal is applied with a repetition rate of at least 50 Hz.

30. The method of claim 20, wherein the at least partial contraction of the deep muscle provides the enhanced appearance of the patient's body by toning the muscle.
